(19) United States
(12) Patent Application Publication
(10) Pub. No.: US 2020/0316096 A1
(43) Pub. Date: Oct. 8, 2020

(54) COMBINATION OF DIET AND DRUG THERAPY FOR TREATING CANCER

(71) Applicant: Trustees of Boston College, Chestnut Hill, MA (US)

(72) Inventors: Thomas N. Seyfried, Foxboro, MA (US); Purna Mukherjee, Boston, MA (US)

(73) Assignee: Trustees of Boston College, Chestnut Hill, MA (US)

(21) Appl. No.: 16/839,369

(22) Filed: Apr. 3, 2020

Related U.S. Application Data
(60) Provisional application No. 62/829,285, filed on Apr. 4, 2019.

(51) Int. Cl. A61K 31/655; A23L 33/00; A61P 35/04

(52) U.S. Cl. CPC A61K 31/655; A61P 35/04; A23L 33/30

(57) ABSTRACT

The present disclosure provides a unique novel diet-drug combination, and method thereof, for cancer management, with synergistic and non-toxic effects.

This Application claims the benefit of U.S. Provisional Application No. 62/829,285, filed on Apr. 4, 2019, which is incorporated herein by reference in its entirety.

### FIELD OF THE INVENTION

The present invention is directed to a combination of diet and drug therapy for treating cancer with synergistic and non-toxic effects.

### BACKGROUND OF THE INVENTION

Glioblastoma multiforme (GBM) has the highest mortality rate among primary brain tumours in adults and remains largely unmanageable with a life expectancy of about 6-24 months following diagnosis. Despite decades of research, little progress has been made in improving GBM survival¹. A defining characteristic of GBM is the ‘secondary structures of Scherer’, which includes diffuse parenchymal growth and invasion over the subpial surface, along white matter tracks, and through the Virchow-Robin space². The highly invasive nature of GBM makes most current therapies ineffective. Most patients die from intracranial pressure due to inflammation, edema, and distal tumour cell invasion. Hence, the proliferative and invasive nature of GBM is the major hindrance for effective therapeutic intervention.

GBM contains a range of morphologically diverse neoplastic cell types that express glial, stem cell, and myeloid/mesenchymal markers³⁻⁵. Also recognized are abnormalities in the number, structure, and function of mitochondria in GBM tumour tissue⁶⁻¹⁰. In addition, recent studies show abnormalities in GBM mitochondrial-associated membranes, which are also critical for normal mitochondrial function¹¹. Moreover, the content and composition of cardiolipin, the signature lipid of the mitochondrial inner membrane that regulates oxidative phosphorylation (OxPhos), shows deviations from the normal in five different murine GBM models¹². As mitochondrial function and efficiency is dependent on structure¹³, OxPhos is expected to be lower in GBM than in normal brain tissue. Indeed, significant evidence shows that OxPhos is defective in human and murine GBM^{7,12,14}.

Regardless of GBM cell type, glucose and glutamine are required for growth and invasion through glycolysis and glutaminolysis, respectively¹⁵⁻¹⁹. Although glucose is the primary metabolic fuel for neurons and glia, normal brain cells can transition to ketone bodies (primarily β-hydroxybutyrate) for energy under hypoglycemic conditions²⁰. In contrast, GBM cells lack metabolic flexibility and are susceptible to death in response to reduced glucose bioavailability²¹. Although brain glutamine levels are tightly regulated via the glutamine-glutamate cycle, the bioavailability of glutamine increases following surgery, radiation, and chemotherapy, which fuels tumour growth^{20,22}. If glucose and/or glutamine remain available, the tumour cells will utilize the fuel and grow, making long-term management difficult.

The calorically restricted ketogenic diet (KD-R) is a low carbohydrate, high fat diet, that reduces blood glucose and increases blood ketone bodies to therapeutic levels, while simultaneously inducing anti-inflammatory effects²³. The primary ketone bodies, β-hydroxybutyrate and acetoacetate, are also neuroprotective and non-fermentable^{24,25}. We found previously that the KD-R is a non-toxic anti-inflammatory approach to reduce pathology in experimental human and mouse brain tumours²³. The neoplastic GBM cells can also synthesize high-energy phosphates through mitochondrial substrate level phosphorylation supported by glutaminolysis^{25,26}. In addition, glutamine provides a continuous source of nitrogen for de novo synthesis of nucleotides and proteins in cancer cells²⁷. The extent of glucose and glutamine metabolism in tumour cells depends not only on the tumour cell type, but also on the location and microenvironment.

Recently, the efficacy of simultaneous inhibition of both glycolysis and glutaminolysis has been demonstrated in experimental cancer models and in a human GBM case report^{15,28,29}. Therapeutic success depends on modifications in dosage, scheduling, and timing in order to enhance efficacy while reducing toxicity⁶. DON (6-diazo-5-oxo-L-norleucine) is a glutamine antagonist that was initially isolated from a Streptomyces broth in 1956. DON blocks multiple glutaminases, thus restricting key metabolites needed for glutaminolysis and the synthesis of nucleotides and proteins³⁰. The anti-tumour efficacy of DON was confirmed in different tumour models, and in humans with various cancers²⁸⁻³⁰.

### SUMMARY OF THE INVENTION

The present disclosure provides a novel unique diet-drug combination with therapeutic synergy for cancer management. In certain embodiments, a cancer patient is on a calorie restricted ketogenic diet (KD-R) and is administered an effective amount of a drug that targets one or more energy metabolite for cancer cells growth and progression. In certain embodiments, the drug is a glucose or glutamine analogue. The present disclosure encompasses any glucose or glutamine analogue, now known or later developed. In certain embodiments, the glutamine analogue is 6-diazo-5-oxo-L-norleucine (DON), and the energy metabolite is glucose, glutamine, or a combination thereof.

The present disclosure provides a major improvement to management of brain cancer and any cancer that uses glucose and glutamine for growth and progression. The present disclosure encompasses all cancers use glucose, glutamine, or some combination of these energy metabolites for growth and progression. Cancer cells are considered reliant on glucose and glutamine for growth and survival. Most drugs that target glutamine are either too toxic or are ineffective in managing cancer growth. DON was therapeutically effective but was considered too toxic for general use against most human cancers. However, the present disclosure provides that, when DON used with the KD-R, DON retains therapeutically efficacy without noticeable toxicity. The present disclosure thus provides a combination of ketogentic diet (KD) together with low doses of DON. In certain embodiments, the present disclosure further provides that this diet/drug combination is the first therapeutically effective, non-toxic treatment for glioblastoma. There are no current therapies available that are effective in managing human glioblastoma multiforme. Hence, the present disclosure provides highly significant and novel management for glioblastoma.

The present disclosure provides an effective non-toxic therapy for patients suffering from glioblastoma multiforme, or any cancer dependent on glucose and glutamine for survival. In certain embodiments, the present disclosure provides that, while the KD-R has shown some therapeutic benefit in managing GBM in some patients, the KD-R alone is not likely to provide long-term management for most people with GBM. Therapeutic benefit is greatly enhanced when the KD-R is used together with DON. Indeed the interaction is synergistic and non-toxic. In addition to GBM, the diet/drug therapy disclosed in the present disclosure provides effective for managing any cancer that uses glucose and glutamine for growth, which includes the majority of cancers, including but not limited to, brain cancer, breast cancer, lung cancer, skin cancer, kidney cancer, and pancreatic cancer. There is currently no cancer therapy that can provide long-term resolution without causing some toxicity. Accordingly, the present disclosure addresses a major need in the field of cancer therapeutics, i.e., a non-toxic metabolic therapy for the majority of cancers, including but not limited to, GBM.

The present invention further provides a method for managing cancer using a diet-drug combination therapy. In certain embodiments, the cancer patient is on the calorie restricted kenogenic diet (KD-R), and the drug is a glucose or glutamine analogue that targets glucose or glutamine or the combination of energy metabolites for cancer cell growth and progression.

### BRIEF DESCRIPTION OF THE FIGURES

The patent or application file contains at least one drawing executed in color. Copies of this patent or patent application with color drawing(s) will be provided by the Office upon request and payment of the necessary fee.

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1F. Restricted Ketogenic diet with DON reduces progression and mortality of the VM-M3 GBM. VM/Dk inbred mice were implanted orthotopically with a small (1.0 mm×1.0 mm) tissue fragment from the VM-M3 tumour on day 0. The implanted mice were divided into two groups on day 4 and were fed either; a standard chow diet unrestricted or ad-libitum (SD-UR), or a ketogenic diet (KD-R) in restricted amounts to reduce body weight by about 15%. DON (0.1-1.0 mg/kg) was injected i.p. 7 days following orthotopic tumour implantation. The diet feeding was continued, and DON was injected every day or every alternate day as shown, FIG. 1A. All mice were imaged in-vivo and terminated on day 14 or 15 when all control mice appeared moribund (experiments 1 & 2). For experiment 1, bioluminescence was scored from 0-4, following the administration of lower doses of DON (0.1 and 0.5 mg/kg). Values are expressed as the mean+/−SEM and a one-way analysis of variance followed by Tukey's post hoc test was performed to determine the significance between groups, FIG. 1B. In experiment 2, in-vivo bioluminescent photon values were obtained following the administration of DON (1.0 mg/kg) in mice under the KD-R, FIG. 1C. The average in-vivo bioluminescent photon values were calculated for the KD-R (n=4) and KD-R+DON (n=8) mice in experiment 2. Values are presented as the mean+/−SEM and the P value was calculated using a two-tailed student's t-test, FIG. 1D. in-vivo bioluminescent images of 3 representative mice from both study groups in experiment 2 are shown, FIG. 1E. For experiment 3, a survival study was performed, and a Kaplan Meier survival plot was configured for SD-UR (n=15), SD-UR+DON (n=10), KD-R (n=10), and KD-R+DON (n=10), FIG. 1F. The log-rank statistical analysis test showed a significant difference between groups in this survival study.

FIGS. 2A-2C. Restricted Ketogenic diet with DON reduces VM-M3 GBM bioluminescence. VM/Dk mice were implanted with a VM-M3 tumour as described in the previous figures. Mouse brains were imaged ex-vivo on day 14 or 15. Mice were imaged ex-vivo after in-vivo bioluminescent imaging, as described in the previous figures. Individual ex-vivo bioluminescent photon values were measured following the administration of DON (1.0 mg/kg) in KD-R mice, FIG. 2A. From the individual measurements, the average ex-vivo bioluminescent photon value was calculated for both the KD-R (n=6) and KD-R+DON (n=8) mouse groups. Values are presented as the mean+/−SEM and significance of differences was determined following a two-tailed student's t-test, FIG. 2B. ex-vivo bioluminescent images of brains from representative mice in experiment 2 are shown in comparison to SD-UR, FIG. 2C.

FIGS. 3A-3C. Restricted Ketogenic Diet with DON kills VM-M3 GBM cells in brain. VM/Dk mice were implanted with VM-M3 cells orthotopically in the brain on day 0 as described in the previous figures. The brains were fixed in formalin for histology, processed, and stained as described in Methods. Histological analysis (H&E) was used to validate the presence of tumour cells, FIG. 3A. The top panel images show the core of the tumour mass growing in the brain. The black boxes in the top panel's images are shown in higher magnification in the bottom panels. Ki-67 positive nuclear staining (100×), as expressed by green fluorescent labeled cells are shown in FIG. 3B. DAPI is used for the Ki-67 negative nuclear stain. The invasion of tumour cells in the brain tissue is evident (arrows) in both histological (H&E) analysis (100×) and Ki-67 staining FIG. 3C.

FIGS. 4A and 4B. Restricted Ketogenic diet increases DON delivery to the VM-M3 GBM. The content of DON in the VM-M3 brain tumour tissue was quantified using two LC/MS/MS procedures, as described in Methods. The brain tissue was analyzed for DON content 60 minutes after i.p. DON injection. The values in experiment a are presented as mean of two independent samples, while the values in experiment b are presented as the mean±SEM (n=3). The difference between the two groups in experiment b was significant (P<0.01). FIGS. 4A and 4B both analytical procedures showed that DON content in tumour tissue was greater under KD-R feeding than under SD-UR feeding.

FIGS. 5A and 5B. Restricted Ketogenic diet with DON reduces TNF-α in the VM-M3 brain tumour tissue, and the Glucose Ketone Index (GKI) in the blood. ELISA was used to measure TNF-α in brain tumour tissue lysates in two different experiments, and the content was expressed as pg/mg of protein. Normal brain (NB) was used as a negative control tissue. The range of TNF-α concentration was −0.04-0.1 pg/mg for NB (n=2 normal mouse brain tissue); 0.4-14.7 pg/mg for SD-UR (n=5 mouse tumour brain tissue); 0.09-11.2 pg/mg for KD-R (n=5 mouse tumour brain tissue); and 0.02-1.0 pg/mg for KD-R+DON (n=7 mouse tumour brain tissue), FIG. 5A. Data showing that blood glucose is lower and blood ketones are higher in mice fed the KD-R (n=10 independent mouse blood samples) than in mice fed the SD-UR (n=10 independent mouse blood samples). This shift in blood glucose and ketones causes a reduction in the GKI, FIG. 5B. Values are expressed as the mean+/−SEM and a one-way analysis of variance followed by Tukey's post hoc test was performed to determine the significance between groups.

FIGS. 6A-6C. Restricted Ketogenic diet with DON reduces the Iba-1 expression in the VM-M3 brain tumour tissue. Immunohistochemistry (IHC) of Iba-1 was performed in formalin fixed brain tissues as described in Methods. In comparison to all other groups, the expression of Iba-1 is highest in the SD-UR tumour as seen by the positively stained brown cells. Iba-1 IHC staining was noticeably less in the VM-M3 tumor from the KD-R-fed mice, and significantly less in the tumour of the DON treated mice, FIG. 6A. Western blot analysis of Iba-1 protein expression in the tumours indicates a decrease in the expression of Iba-1 in the DON treated tumours (n=3 mouse tumour brain tissue) in comparison to SD-UR (n=3 mouse tumour brain tissue). A decrease in expression was also seen for KD-R mice (n=2 mouse tumour brain tissue). Normal brain (NB) was used as a negative control tissue, FIGS. 6B and 6C.

FIG. 7A-7D. Restricted Ketogenic diet with DON reduces progression and mortality of the CT-2A GBM. C57BL/6J mice were implanted with CT-2A tumour fragments on day 0, as described in FIGS. 1A-1F. Brain wet weights were measured for SD-UR (n=4 mouse brains), KD-R (n=7 mouse brains), and KD-R+DON (n=5 mouse brains), FIG. 7A. Ex-vivo bioluminescent photon values were calculated for the same brain samples and presented as the mean±SEM, FIG. 7B. All brains were imaged on day 14 or 15. A one-way analysis of variance followed by Tukey's post hoc test was performed to determine the significance between groups. Representative brain sample images from each study group were portrayed in FIG. 7C. A Kaplan Meier survival curve was plotted for the SD-UR (n=6 mice), SD-UR+DON (n=8 mice), KD-R (n=6 mice), and KD-R+DON (n=8 mice) groups FIG. 7D. The log rank test was used to determine the significance.

FIG. 8. Targeting Glucose and Glutamine using KD-R with DON for the Metabolic Management of the VM-M3 and CT-2A Experimental GBM. GBM tumour cells are largely dependent on glucose and glutamine for survival and growth. Energy through substrate level phosphorylation (SLP) in the cytoplasm (glycolysis) and in the TCA cycle (glutaminolysis) will compensate for reduced energy through oxidative phosphorylation (OxPhos) or hypoxia that occurs in these GBM cells. The KD-R will reduce glucose carbons for both the glycolytic and pentose phosphate (PPP) pathways that supply ATP and precursors for lipid and nucleotide synthesis, as well as for glutathione production. DON will inhibit glutaminases thus depleting glutamate and the glutamine-derived amide nitrogen for ammonia and nucleotide synthesis. Depletion of glutamine-derived glutamate will reduce anapleurotic carbons to the TCA cycle through α-KG for protein synthesis, while also reducing ATP synthesis at the succinyl CoA synthase step in the TCA cycle⁴¹. The glutamine-derived glutamate is also used for glutathione production that protects tumor cells from oxidative stress. The KD-R+DON will thus make the VM-M3 and the CT-2A cells vulnerable to oxidative stress. The simultaneous targeting of glucose and glutamine using the KD-R+DON will starve tumour cells of energy production while blocking their ability to synthesize proteins, lipids, and nucleotides. This metabolic starvation could also reduce extracellular acidification through reduction of lactate and succinate. The elevation of non-fermentable ketone bodies will provide normal cells with an alternative energy source to glucose while also protecting them from oxidative stress. This diagram and legend have been modified from that presented previously⁶.

FIGS. 9A-9H. Invasive behavior in brain of VM-M3 GBM tumour cells. VM-M3/Fluc tumor fragments were implanted as described in FIG. 1A-1F. Histological analysis (H&E) was used to validate the presence of tumour cells in the different regions of the brain as indicated in the figures. A defining characteristic of GBM is the secondary structures of Scherer, which is evident in panels A-H. FIG. 9A: tumour cell infiltration in the brain (scale bar=175 μm). FIG. 9B: core tumor area (scale bar=300 μm). FIG. 9C: tumor (T) and necrotic (N) areas (scale bar=525 μm). FIG. 9D: necrosis in higher magnification (scale bar=140 μm). FIG. 9E: arrows indicate atypical mitosis (scale bar=100 μm). FIG. 9F: arrows show perivascular invasion (scale bar=65 μm). FIG. 9G: arrows show perivascular invasion and circle shows sub-arachnoid invasion (scale bar=200 μm). FIG. 9H: subarachnoid space infiltration in higher magnification (scale bar=65 μm).

FIG. 10. Histological analysis of KD-R and DON treated CT-2A tumour brains. H&E was used to validate the influence of the KD-R and DON treatment on the CT-2A tumor cells. Images show the tumor core at 200× (top), and at 400× (bottom). Arrows indicate nuclear mitotic arrest. All scale bars are 100 μm for 200× and 25 μm for 400×.

FIG. 11. Body weights of VM-M3 tumour bearing mice. VM-M3/Fluc tumour fragments were implanted as described in FIGS. 1A-1F. Body weight was determined every alternate day. Initial and final body weight data are presented. Mice fed the SD-UR began to drop body weight on the day of termination due to tumour burden. Mice fed the KD-R maintained a 15-18% body weight reduction throughout the experiment due to reduced caloric intake. Initial and final body weights were similar in the SD-UR and the SD-UR+DON mice (n=15 mice/group). Final body weights were lower than initial body weights in the KD-R and the KD-R+DON mice (n=15 mice/each group). Values are expressed as the mean+/−SEM and a two tailed student's t-test was performed between the initial and final values of each group (*=p<0.01).

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION OF THE INVENTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a dendrite,” “an antibody,” or “a biosensor,” includes, but is not limited to, two or more such dendrites, antibodies, biosensors, and the like, including a plurality of such dendrites, antibodies, biosensors, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an antibody refers to an amount that is sufficient to achieve the desired improvement or effect modulated by indicated component, material, compound or protein, e.g. achieving the desired level of binding with an analyte bound by the antibody. The specific level in terms of concentration or amount as an effective amount will depend upon a variety of factors avidity of the antibody, target analyte, desired level of assay sensitivity and the like.

Reference to “a/an” chemical compound, protein, and antibody each refers to one or more molecules of the chemical compound, protein, and antibody rather than being limited to a single molecule of the chemical compound, protein, and antibody. Furthermore, the one or more molecules may or may not be identical, so long as they fall under the category of the chemical compound, protein, and antibody. Thus, for example, “an” antibody is interpreted to include one or more antibody molecules of the antibody, where the antibody molecules may or may not be identical (e.g., different isotypes and/or different antigen binding sites as may be found in a polyclonal antibody).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as those referred to herein below.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

As used herein, the terms “optional” or “optionally” means that the subsequently described event or circumstance can or cannot occur, and that the description includes instances where said event or circumstance occurs and instances where it does not.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

Unless otherwise specified, temperatures referred to herein are based on atmospheric pressure (i.e. one atmosphere).

From the foregoing, it will be seen that aspects herein are well adapted to attain all the ends and objects hereinabove set forth together with other advantages which are obvious, and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only and is not intended to be limiting. The skilled artisan will recognize many variants and adaptations of the aspects described herein. These variants and adaptations are intended to be included in the teachings of this disclosure and to be encompassed by the claims herein.

The present disclosure provides a unique novel diet-drug combination for managing cancer with synergistic and non-toxic effects. In certain embodiments, the diet is a calorie restricted ketogenic diet (KD-R), and the drug is for targeting energy metabolite, such as glucose or glutamine, or the combination of these energy metabolites, for cancer cell growth and progression. In certain embodiments, the drug is glucose or glutamine analogues. In certain embodiments, the glutamine analogue is 6-diazo-5-oxo-L-norleucine (DON). The present disclosure encompasses all glucose and glutamine analogues, now known or later developed.

The present disclosure further provides methods for managing or treating cancer using the diet-drug combination. The cancer includes all majority of cancers that uses glucose and/or glutamine for cancer cell growth and progression. Such cancers include, but are not limited to, brain cancer, breast cancer, lung cancer, skin cancer, kidney cancer, and pancreatic cancer. In certain embodiments, the present disclosure provides a method for treating brain cancer, such as glioblastoma (GBM), human glioblastoma multiforme, using the diet-drug combination.

In certain embodiments, the present disclosure provides a unique therapeutic response from the simultaneous targeting of glucose and glutamine using a KD-R and DON, respectively, in syngeneic orthotopic mouse models of GBM. The VM-M3 GBM tumour arose in the cerebral cortex of a mouse from the VM/Dk inbred strain, which has a high incidence of spontaneous brain tumours relative to other strains. The mesenchymal VM-M3 tumour cells invade distally throughout the brain using the “secondary structures of Scherer”². In addition to the VM-M3 tumour, the present disclosure provides the effects of the KD-R and DON combination on the growth of the CT-2A brain tumour. The highly angiogenic CT-2A tumour was produced from 20-methancolanthrene in the cerebral cortex of a C57BL/6J mouse, and has several characteristics in common with the neoplastic neural stem cells found in human GBM^{31,32} Abnormalities in the electron transport chain were found in both tumours¹². These data show that the combination of KD-R and DON administration cause massive tumour cell death in both of these preclinical GBM mouse models and highlights the importance of targeting glycolysis and glutaminolysis simultaneously for the metabolic management of GBM.

Therefore, in more details, the present disclosure provides the therapeutic action of glucose and glutamine targeting in the VM-M3 and CT-2A syngeneic mouse models of GBM. It is known that calorie restriction, ketogenic diets, and DON can reduce metabolites in the glycolytic and glutaminolysis pathways, respectively^{33,39-41} However, it was showed that the KD-R+DON could rescue mice with these GBM tumours from late stage orthotopic growth. The diet/drug therapy significantly reduced brain swelling, hemorrhage, and morbidity. The KD-R alone reduced the VM-M3/Fluc cell invasion and proliferation as seen by both in-vivo and ex-vivo bioluminescent imaging of the brains, with Ki-67 staining, and with histology of the brain tissues. Moreover, the KD-R produced therapeutic ketosis (low GKI value) and reduced TNF-α and Iba-1 levels, suggesting reduced inflammation as compared to the SD-UR control brains. The ability of DON treatment to arrest tumour growth and promote survival late in disease progression suggests a potentially direct role for glutamine metabolism in VM-M3/Fluc growth and invasion. The present disclosure provides the finding that is consistent with the importance of glutamine in the metabolism of mesenchymal cells from which the VM-M3 tumour is derived^{4,37,42}. Although DON treatment alone was effective in reducing tumour growth and invasion in the control SD-UR mice, a synergistic effect was observed when DON was administered together with the KD-R, providing evidence for a significant diet/drug interaction. The present disclosure further supports the press-pulse therapeutic strategy for the metabolic management of cancer with the KD-R serving as a press therapy and DON serving as a pulse therapy⁶.

Interestingly, the level of DON in the brain tumour tissue was significantly higher under KD-R feeding than under SD-UR feeding suggesting that the KD-R facilitates DON delivery to the brain. This is significant because it allowed for a lower therapeutic dose of DON when administered under a KD-R regimen. A similar observation was made previously in showing that a KD-R facilitated the delivery of the iminosugar, N-butyldeoxynojirimycin, across the blood brain barrier in mice with Sandhoff disease⁴³. Although the mechanism remains to be determined, these findings suggest that the KD has potential as a facilitator of drug delivery to the brain for a broad range of neuro-pathological conditions. It is also important to mention that the previous findings clearly demonstrated that caloric restriction can maintain the blood vessel integrity in the mouse CT-2A and the human U-87 GBM and thus reduce the leakiness of the neovasculature⁴⁴. Interestingly, a recent observation by Gordon et al. showed that DON treatment promotes the recovery of blood-brain-barrier integrity in the mouse model of cerebral malaria, though the mechanism remains unclear³³. More studies are needed to clarify the mechanism by which a KD-R facilitates drug delivery to the brain.

DON has shown therapeutic efficacy in human cancers of the blood, colon, and lung, but issues of toxicity were noted in some cases³⁰. No noticeable toxicity was seen in the DON treated mice until the end of the study when mild muscle loss was observed in some of the mice. It is proposed that issues with DON toxicity arise when there is an appreciable competition for glutamine between host and tumour. There is also increasing evidence supporting the protective role of glutamine supplementation for cancer patients⁴⁶. Since tumour consumption of glutamine is dissipative, glutamine supplementation might not increase tumour growth⁴⁶. However, additional preclinical studies are needed to determine if glutamine supplementation could be used together with DON. The present disclosure provides that strategies for managing toxicity of a therapeutically effective drug would be as important as developing a new drug. The present results indicate that the KD-R enhances the therapeutic action of DON, thus reducing dosage and toxicity.

The therapeutic action of the diet/drug effect seen in the VM-M3 tumour was also seen in the CT-2A neural stem cell tumour. As observed from histological analysis, the KD-R+DON treatment caused massive mitotic arrest or catastrophe in the CT-2A brain tumour cells. The results show that therapeutic efficacy against orthotopic brain tumour growth was significantly greater using the diet/drug combination than in using either the KD-R or DON alone. The CT-2A tumour shares several characteristics with glioma stem cells³², whereas the VM-M3 tumour shares several characteristics with the invasive mesenchymal cell found in most GBM². Hence, these findings suggest that the KD-R+DON therapeutic strategy could potentially target the two most common neoplastic cell types found in human GBM³. The recent human GBM case report provides support for this prediction¹⁵.

Several mechanisms could underlie the therapeutic action of the diet/drug therapy used to treat the VM-M3 and CT-2A tumours. The KD-R simultaneously targets the glycolytic and pentose phosphate pathways, which are upregulated in GBM⁴⁷⁻⁴⁹. It was previously showed that calorie restriction and restricted ketogenic diets target the IGF-1, PI3K, AKT, and Hif-1a signaling pathways in the CT-2A tumour^{38,50}. The down-regulation of these pathways would reduce angiogenesis, inflammation, and mTOR signaling, while enhancing tumour cell apoptosis^{23,44,51,52}. As glucose is the fuel for glycolysis, the pentose phosphate pathway, and serine biosynthesis, the KD-R could reduce multiple growth metabolites, tumour cell glutathione levels, and nucleotide synthesis. Furthermore, the KD-R also reduces one-carbon metabolism especially in glioma cells lacking OxPhos capacity^{25,49,53}. Hence, the glucose-restricting action of the KD-R could target multiple signaling pathways linked to glioma growth and progression.

In addition to restricting glucose availability, the KD-R could also elevate β-hydroxybutyrate and acetoacetate, the major circulating ketone bodies produced in the liver, from the active metabolism of medium chain triglycerides⁵⁴. Ketone bodies increase the redox span of the CoQ couple, thus reducing oxidative stress in normal brain cells⁵⁴. These ketone bodies are also neuroprotective and have beneficial therapeutic value for various diseases^{24,55}. As ketone bodies are non-fermentable and require efficient OxPhos for generating ATP^{25,54}, the VM-M3 and CT-2A cells are unable to metabolize these metabolites for energy due to insufficient OxPhos 12,25. Ketone bodies cannot be used as an alternative energy fuel in cells with defective mitochondria^{12,25}. Previous studies demonstrated that glioma cells cannot use ketone bodies and that ketone bodies do not stimulate tumour growth^{56,57}. Hence, the KD-R has a dual function in 1) targeting glucose-dependent signaling pathways that drive tumour growth, and 2) in providing an alternative metabolic fuel to normal brain cells under glucose restriction.

While the KD-R reduces metabolites through glycolysis and the pentose phosphate pathway, DON blocks glutaminolysis, thus depriving the tumour cells of the amide nitrogen needed for nucleotide and protein synthesis, and at the same time, depletes the glutamate needed for synthesis of α-ketoglutarate (α-KG)^{25,58,59}. α-KG is a precursor for lipid synthesis through reductive carboxylation and is the substrate for ATP synthesis through the succinate-CoA ligase reaction in the TCA cycle under hypoxia^{25,60}. As mentioned previously, however, glutamine targeting is more challenging than is glucose targeting due to the importance of glutamine for the immune system and the gut^{6,61}. Consequently, glutamine targeting must be done strategically to avoid damage to those systems that are needed for normal physiological function^{6,15}. It is for this reason that DON was administered to the tumour-bearing mice at low doses and transiently. DON was administered 7 days after tumour implantation, which is considered late for the VM-M3/Fluc tumour because neurological signs and symptoms start to appear 10 to 12 days post implantation followed by 100% death in 14 to 18 days. The late administration of DON was chosen to ensure adequately high ketone levels were achieved in order to facilitate neuroprotection and to reduce tumour inflammation. Secondly, the late treatment could serve as a comparable model when treating late stage GBM patients. A 60 to 70 percent reduction in tumour growth was observed when DON was administered at 0.5 mg/kg once or twice per week. In comparison, a near complete resolution of the brain tumour was achieved when 1.0 mg/kg of DON was administered with the same schedule, as seen with bioluminescent imaging. These results are significant because no other clinical or pre-clinical study using DON considered a KD-R as a method for protecting the host from toxicity. Case reports of GBM and other metastatic cancers show the significance of the KD-R when combined with other therapies^{15,62,63}.

These findings support previous suggestions that reduced glutaminolysis would be a key therapeutic action of DON against the growth of glutamine-dependent tumours, including GBM^{29,30}. Although it is known that DON targets glutaminolysis in tissues^{30,33,58} and that reduced levels of circulating glucose reduces glycolysis^{64,65}, it was unable to find consistent brain metabolite changes in pathways of glycolysis and glutaminolysis in a preliminary metabolomic analysis of KD-R+DON-treated VM-M3 mouse brains. Due to the confounding variables in the in-vivo environment (dead tumour cells, necrotic tissue, and various types of host infiltrating cells), an in-vitro analysis in defined media could provide a clearer picture of the changes in metabolites of the glucose and glutamine pathways due to DON or other drug treatments in the VM-M3 and CT-2A cells. It is also important to determine if other glutaminolysis inhibitors, e.g., epigallocatechin-3-gallate (EGCG), bis-2-(5-phenylacetamido-1,2,4-thiadiazol-2-yl) ethyl sulfide (BPTEs), and CB-839^{19,66}, can match the therapeutic action of DON against brain cancer when used together with the KD-R. Pretreatment with a ketogenic diet before implementing the standard of care is suggested in order to protect normal brain cells from treatment toxicities²².

The findings in the VM-M3 and CT-2A syngeneic GBM models might be considered contradictory to some previous studies in GBM patients and in experimental systems⁶⁷⁻⁶⁹. Previous studies from Bachoo and colleagues suggest minimal ¹³C-glutamine anaplerosis in GBM implying that glutamine restriction in this context may not be therapeutic⁶⁸. As DON inhibits multiple glutaminases, it is possible that DON does not directly impact glutamine anaplerosis in vivo, but acts on other related pathways and systems^{29,30,33}. It was recently described how glutamine is the only amino acid not requiring an energy investment to generate ATP through mitochondria substrate level phosphorylation (mSLP)²⁵. Tardito et al., found that under Gln starvation, intracellular oleate was unaffected, and glucose-dependent glutamate production increased, implying that the contribution of Gln to growth is largely independent of anaplerosis⁶⁹. Further, Oizel et al., identified two clusters of GBM that are Gln^{high} and Gln^{low} based on glutamine utilization¹⁶. These investigators also mentioned that the most aggressive GBM contained mesenchymal cells that utilized the highest levels of glutamine. The VM-M3 cells are highly invasive and of mesenchymal origin^{2,37}. Nevertheless, the present disclosure provides interpretation of the data cautiously, as the metabolic effects of DON's action in killing the GBM cells were inferred rather than measured directly.

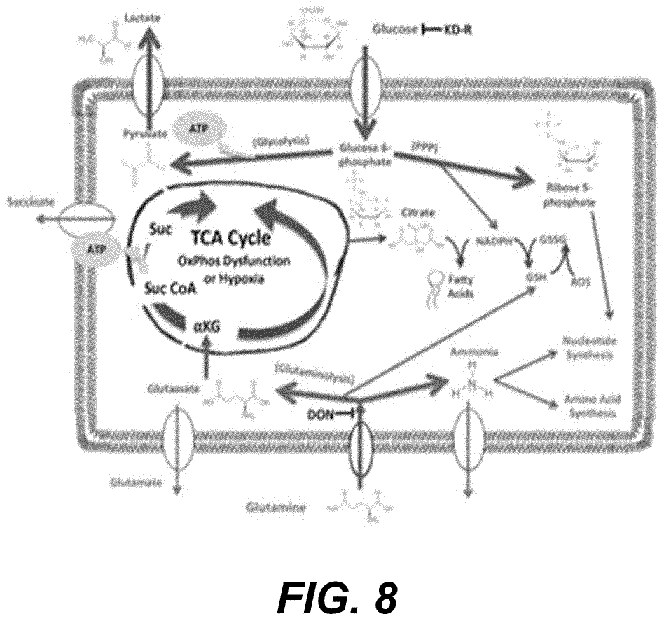

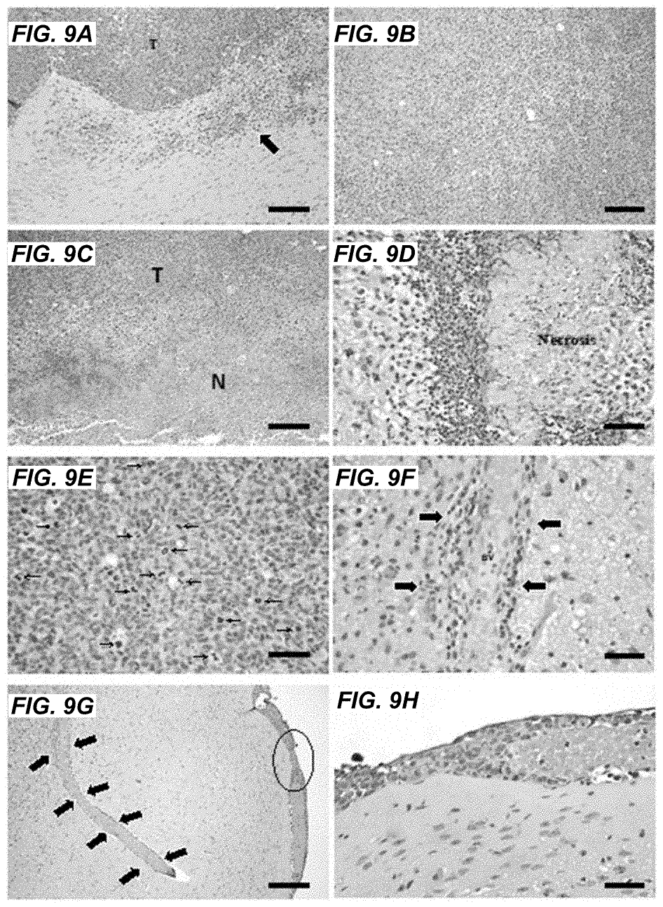

In summary, the present disclosure provides the benefits of targeting glutamine under a KD-R for managing preclinical GBM. The influence of targeting both glucose and glutamine on the metabolic pathways involved with GBM growth is shown in FIG. 8. This treatment was capable of arresting the growth of tumour cells and in promoting the survival of mice with two different syngeneic GBM tumours grown orthotopically. As the mice used for these studies were not treated with surgery, radiation, or standard chemotherapy, a similar therapeutic response may or may not be seen in GBM patients using this approach together with current standard of care¹⁵. Because of the high mortality following a GBM diagnosis, these findings could have clinical implications especially in light of the recent case report¹⁵. Furthermore, these studies reveal a potential role for the ketogenic diet in facilitating drug delivery to brain tumours.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### B. Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The following examples are provided to illustrate embodiments of the present invention but are by no means intended to limit its scope.

The examples described herein will be understood by one of ordinary skill in the art as exemplary protocols. One of ordinary skill in the art will be able to modify the below procedures appropriately and as necessary.

The following studies were to determine if the simultaneous targeting of glucose and glutamine, while under therapeutic ketosis, could manage late stage growth and enhance survival without toxicity in two different syngeneic mouse models of GBM. DON was administered 7 days after tumour implantation, which is considered late for the VM-M3/Fluc tumour, as neurological signs and symptoms first appear at 10 to 12 days post implantation with morbidity occurring in 100% of the tumour-bearing mice by 14-18 days². Additional results were obtained in C57BL/6J inbred mice bearing the syngeneic CT-2A high-grade stem cell glioma³². DON, at the concentrations used in this study, had no toxic effects in non-tumour-bearing VM/Dk mice, as was shown previously for C57BL/6J mice³³.

To observe the effect of the KD-R and DON on the VM-M3 tumour model, three consecutive experiments were conducted. Experiment 1 included four groups (SD-UR; SD-UR+DON; KDR; KDR+DON) with lower DON dosages (0.1 and 0.5 mg/kg). Experiment 2 involved both in-vivo and ex-vivo imaging of two study groups (KD-R and KD-R+DON) following the administration of a higher dose of DON (1.0 mg/kg). Experiment 3, a survival study, also used the higher DON dose (1.0 mg/kg) and included the four groups from Experiment 1 (SD-UR; SD-UR+DON; KDR; KDR+DON) ( ).

### Example 1

### KD-R+DON Reduces VM-M3 Tumour Burden and Increases Survival

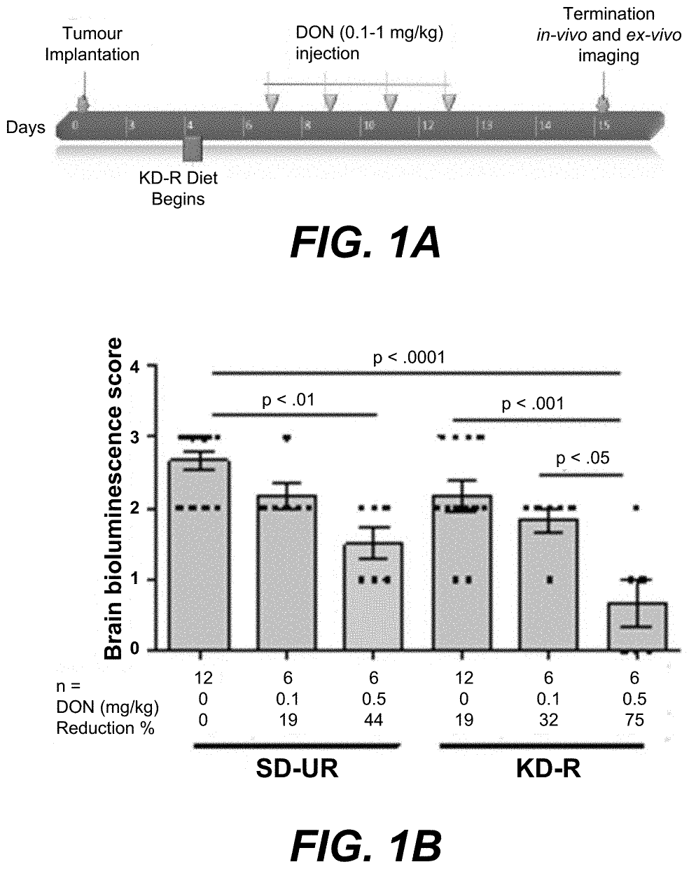

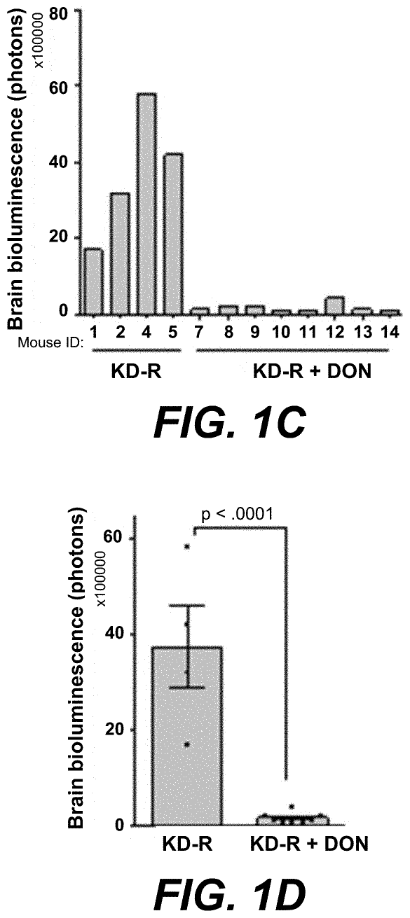

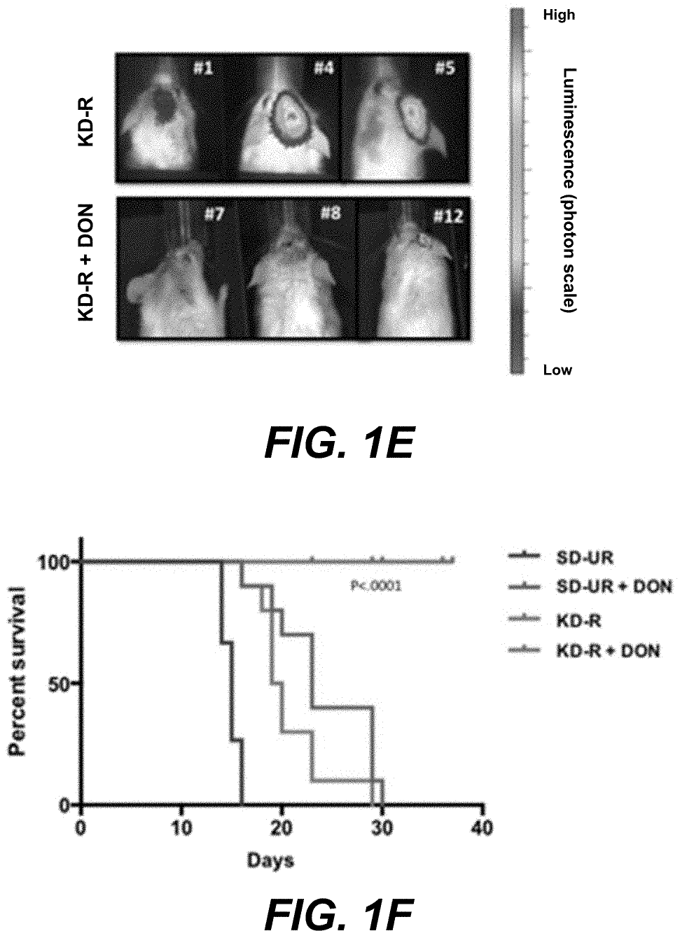

The KD-R+DON treatment was performed as outlined in the study timeline (FIG. 1A). In experiment 1, two doses of DON (0.1 and 0.5 mg/kg) were used in both the SD-UR and KD-R groups (FIG. 1B). d-luciferin was injected i.p. 15 days after tumour implantation, and in-vivo bioluminescence imaging was performed. The total VM-M3 tumour bioluminescence photon values were scored from 0 to 4, with 0 being the baseline value and 4 being the highest value. Several mice from the SD-UR group that did not receive DON had a maximum photon score of 4, whereas the mice from the KD-R group+DON had a minimum photon score of 0 or 1. Compared to the control SD-UR group, mice receiving the KD-R alone or SD-UR+DON had a 19% and 44% lower photon value, respectively (FIG. 1B). The bioluminescence photon score was 75% lower in mice that received the KD-R+DON than in mice receiving each therapy alone, indicating a synergistic effect of the diet/drug combination.

Experiment 2 involved a larger number of mice that were treated with a higher dose of DON (1.0 mg/kg). Due to the observed synergy between the KD-R and DON in experiment 1, only the KD-R group was selected for further experiments. All mice in this experiment expressed a significant amount of tumour burden as observed by in-vivo imaging on day 7 (data not shown). Individual brain bioluminescent values are shown in FIG. 1C. A baseline value of bioluminescence was seen in 7 out of the 8 KD-R fed mice that received DON. A significant reduction in average photon value was observed in the DON treated mice when analyzed statistically (FIG. 1D). A representative number of mice from both study groups are shown in FIG. 1E. All untreated SD-UR control mice died between day 15 and 18. In contrast, mice treated with the diet/drug combination survived until day 40 and onwards (FIG. 1F).

A standard diet restricted (SD-R) group or a ketogenic diet unrestricted (KD-UR) group were not included in this study. It was previously showed that a KD-R is more effective at reaching the target level of blood glucose and ketones than is a SD-R^{23,34,35}. In other words, the ketone levels are higher, and the glucose levels are lower under KD-R feeding than under SD-R feeding despite similar caloric restriction and reduced body weight. A KD-UR was also not included in this study, as consumption of ketogenic diets in unrestricted amounts can cause insulin insensitivity and weight gain, as it was previously described^{23,35,36}.

### Example 2

### KD-R+DON Reduces Ex-Vivo Bioluminescence in VM-M3 Brain

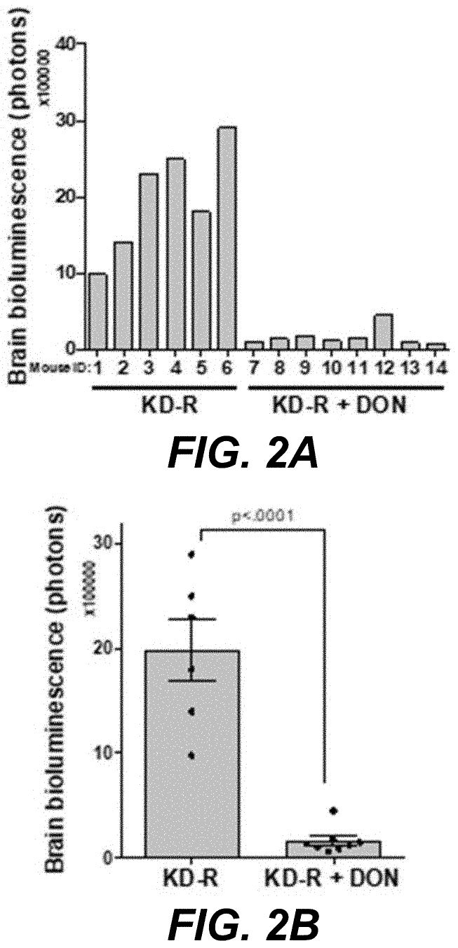

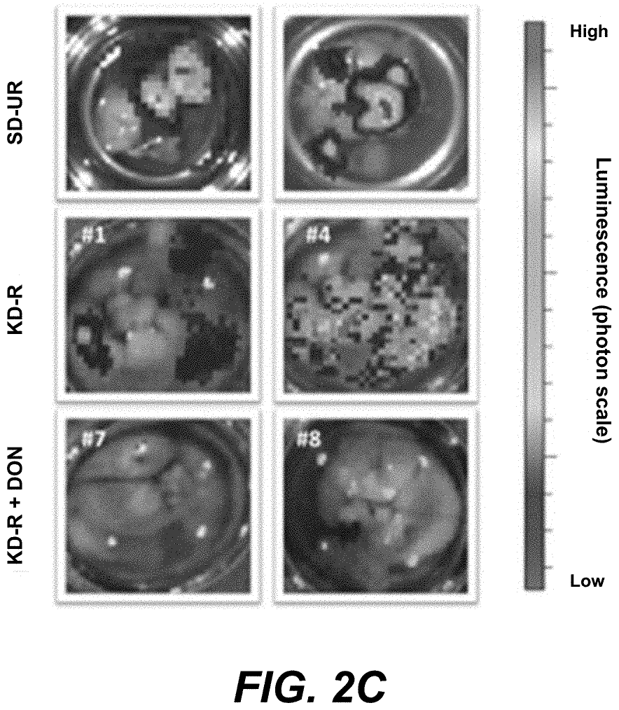

The VM mice from experiment 2 were sacrificed after in-vivo imaging, and their brains were immediately resected and individually imaged ex-vivo for total bioluminescence as described in the Methods below. Ex-vivo imaging provides a more accurate assessment of tumour progression than in vivo brain imaging due to the direct contact of the d-luciferin with the brain tissue and the removal of the blood-brain barrier as a limiting factor. The individual ex-vivo bioluminescent values for the DON treated mice were below the baseline value indicating a significant reduction in tumour cell viability (FIG. 2A). These values were consistent with the in-vivo photon values (FIG. 1C). A significant reduction in average ex-vivo photon value was also observed in the mice treated with DON alone (FIG. 2B). A representative number of brains from the KD-R and the DON-treated mice are shown in comparison to the SD-UR mice (FIG. 2C). It should be noted that an accurate in-vivo bioluminescent value was not obtained for mouse #3 and #6 due to several variables during the imaging process (tumour burden, isoflurane exposure, blood collection, etc.). If an individual mouse did not provide an accurate in-vivo bioluminescent reading, the brain was immediately resected, placed in sterile PBS and imaged ex-vivo. For this reason, there are slight discrepancies in the number of mice imaged for in-vivo bioluminescence and for ex-vivo bioluminescence in experiment 2.

### Example 3

### KD-R+DON Reduces Proliferation and Kills VM-M3 Tumor Cells

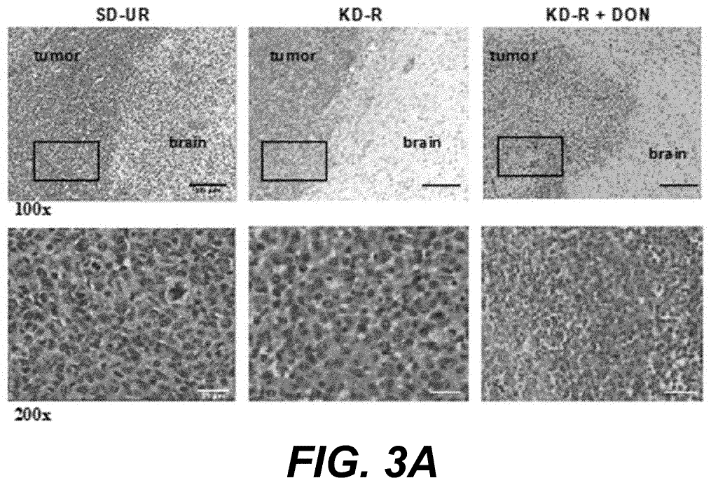

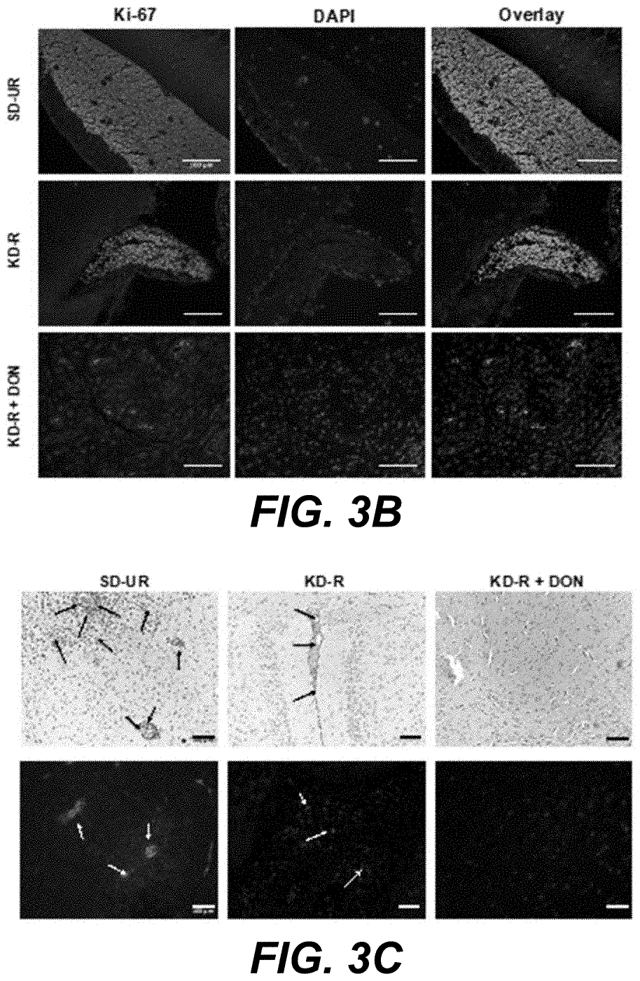

Histological evaluation of brain tumour tissue from mice with the VM-M3 tumour is shown in FIGS. 3A and 3B. Compared with the diffuse, ill-defined border of the VM-M3 tumour cells growing in the SD-UR brains, the tumour in the KD-R brains appeared less dense, less invasive, and had a more defined border (FIG. 3A). DON treated brain tumours showed a significant area of dead cells in the primary tumour. In addition, the percentage of Ki-67 proliferating cells (green) was lower in the KD-R tumour than in the SD-UR tumour and was further reduced in the KD-R+DON treated tumour tissue (FIG. 3B).

VM-M3 cells are highly invasive through the secondary structures of Scherer². VM-M3 cells were observed invading from the core tumour into the normal appearing brain (FIGS. 1A-1F). Moreover, the extensive perivascular necrosis and subarachnoid invasion, hallmarks of human GBM (S1), were clearly present in the VM-M3 model of GBM. The KD-R reduced the invasion of VM-M3 cells as shown in both H&E and Ki-67 staining (FIGS. 3B and 3C). VM-M3 GBM tumour cells were seen invading the perivascular area in the SD-UR brain, whereas fewer invading GBM tumour cells were seen in the KD-R brains. No invading VM-M3 cells were seen in the KD-R+DON treated brains (FIG. 3C). These findings indicate that the diet/drug therapy both kills tumour cells and inhibits invasion.

### Example 4

### KD-R Facilitates DON Delivery to the VM-M3 Tumour

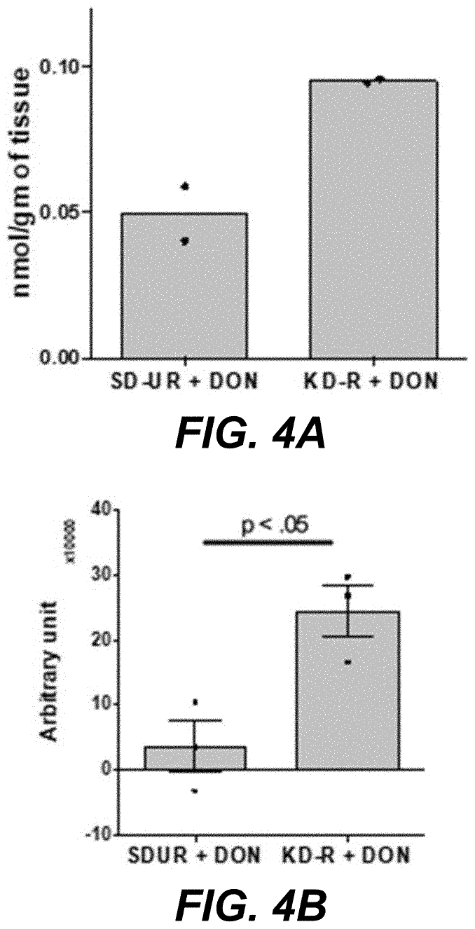

Plasma and brain tissues were prepared for DON analysis one hour after systemic DON injection, as described in Methods below. LC/MS/MS analysis showed that the concentration of DON in the VM-M3 tumour tissue was about 2-fold greater in mice fed the KD-R than in mice fed the SD-UR (FIG. 4A). In another independent experiment, using a similar analytical system, the concentration of DON in the VM-M3 tumour tissue was about 3-fold greater in mice fed the KD-R than in mice fed the SD-UR (FIG. 4B). In both methods of measurement, DON delivery to the brain was greater in the mice fed the KD-R than in the mice fed the SD-UR (FIGS. 4A and 4B). DON was detected in plasma 10 min after i.p. injection (data not shown) and was detected in brain tissue 60 min following injection. These findings indicate that the KD-R facilitated delivery of DON to the VM-M3 tumour tissue.

### Example 5

### KD-R+DON Reduces TNF-α Expression and GKI

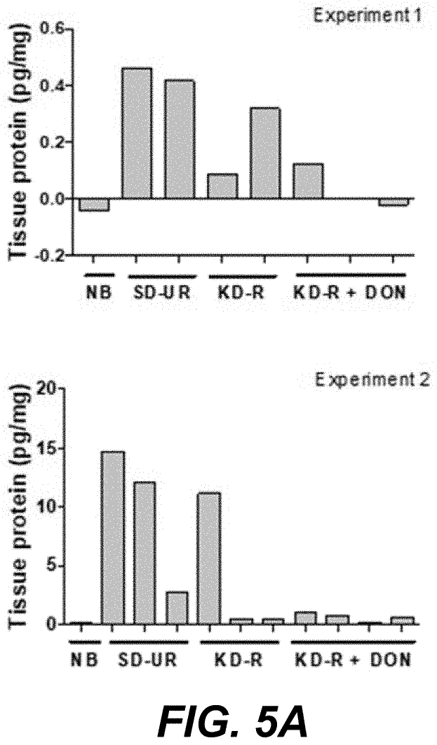

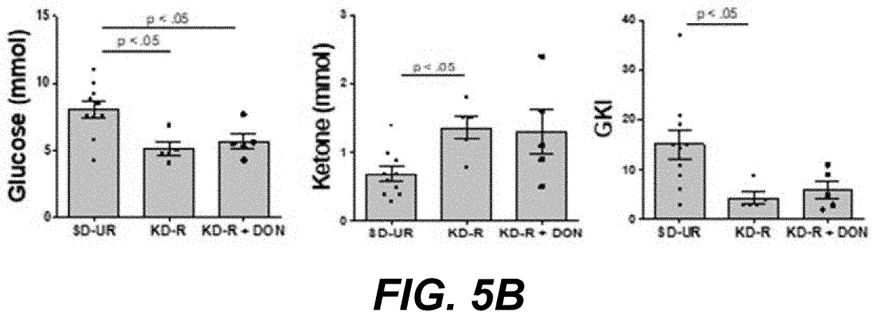

TNF-α expression was measured in lysates prepared from the right brain cortex that contained the VM-M3 tumour. These mouse brain samples are different from the mice used for imaging in FIGS. 1A-1F and 2A-2C. A high level of TNF-α expression, a biomarker for inflammation, was found in the right brain cortex of mice fed the SD-UR. Two independent analyses showed a similar trend in the TNF-α level (pg/mg of protein in the lysate). TNF-α expression was either low or undetectable in the normal brain tissue (FIG. 5A). TNF-α expression was lower in the brain of KD-R-fed mice than in the brain of SD-UR-fed mice, whereas TNF-α expression was even lower in the KD-R+DON treated mice, suggesting a minimum amount of tumour load and reduced inflammation compared to the other two groups (FIG. 5A).

Reduced glucose and increased ketone body levels are blood biomarkers for the KD-R and evidence of therapeutic ketosis. The ratio of glucose to ketone bodies (Glucose Ketone Index, GKI) has been linked to the therapeutic efficacy of the ketogenic diet (KD) for brain cancer³⁶. The lower the GKI value, the greater the metabolic stress on the tumour cells. A significant difference in blood glucose and ketone levels was observed between KD-R and SD-UR mice (FIG. 5B). The GKI values for the KD-R mice, treated and untreated with DON, were significantly lower than for SD-UR mice. The GKI values in the KD-R+DON mice were similar to that in the KD-R mice, indicating that a low GKI was maintained under DON treatment.

### Example 6

### KD-R+DON Reduces Iba-1 Expression in the VM-M3 Tumour

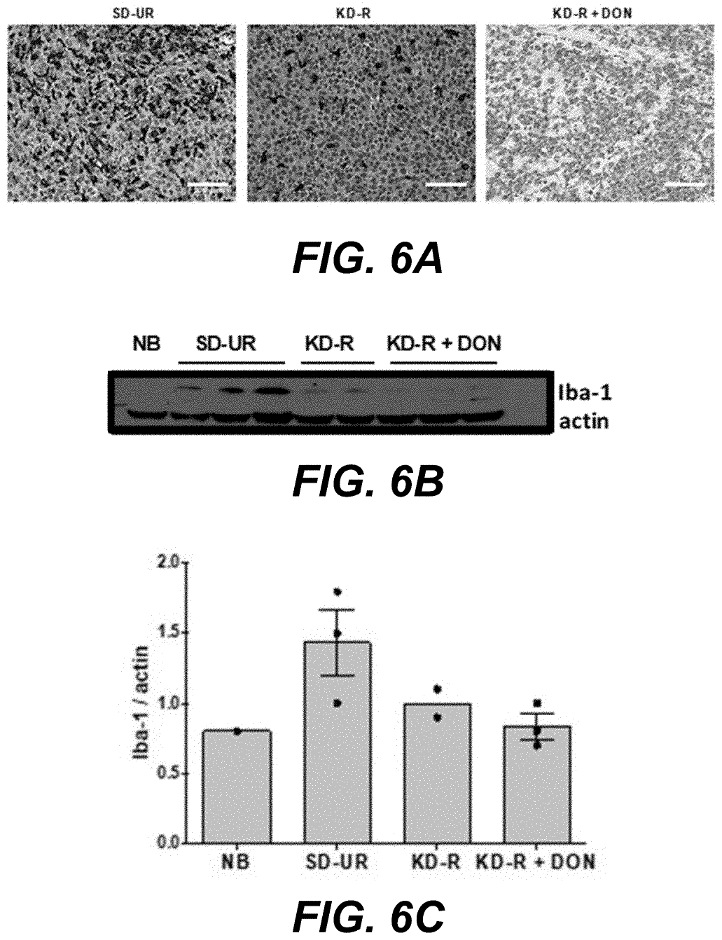

Iba-1 protein expression was analyzed in the VM-M3 brain tumour tissue by immunohistochemistry and western blot. Iba-1 was heavily expressed in VM-M3 tumour and normal microglia/macrophages, as we described previously³⁷. The KD-R reduced the expression of Iba-1 as shown in both immunohistochemistry and western blot (FIGS. 6A and 6B). There was a significant reduction in Iba-1 expression in DON treated brain tumour tissue. It is important to note that the majority of tumour cells in the DON treated tumours were dead and therefore did not stain positively for nuclear methyl green (FIG. 6A). This finding is consistent with the histological observation found from our H&E analysis (FIGS. 3A-3C). The reduced Iba-1 staining is also consistent with the reduced TNF-α expression (FIGS. 5A and 5B), suggesting reduced inflammation in the brains of both KD-R and DON-treated mice. The reduced Iba-1 histological staining was also consistent with the findings from western blot analysis of Iba-1 expression (FIG. 6B). Expression of Iba-1 in the brain tissue of the KD-R+DON treated mice was significantly lower than the expression in the untreated SD-UR mice, and was similar to the expression in normal non-tumour mouse brain. Iba-1 expression was also lower in the KD-R mice than in the SD-UR mice, but the reduction was not significant (FIG. 6B).

### Example 7

### KD-R+DON Reduces Edema in CT-2A Brain and Increases Survival

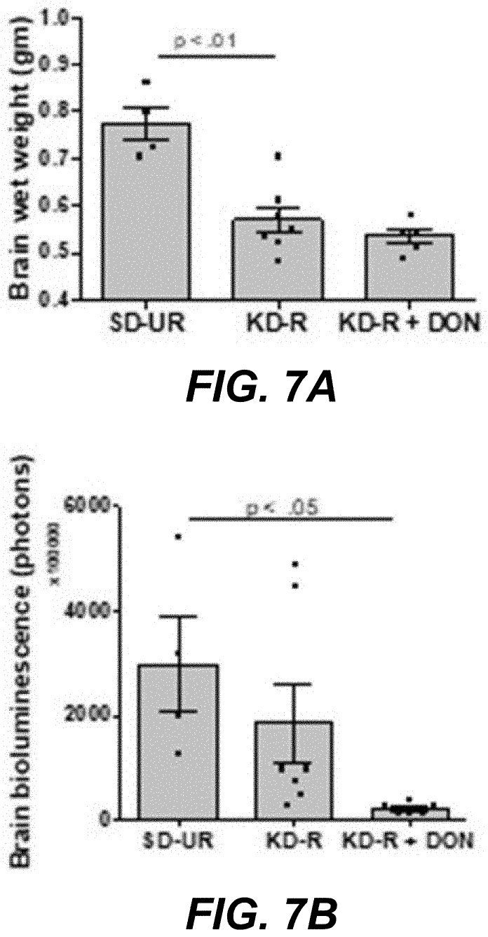

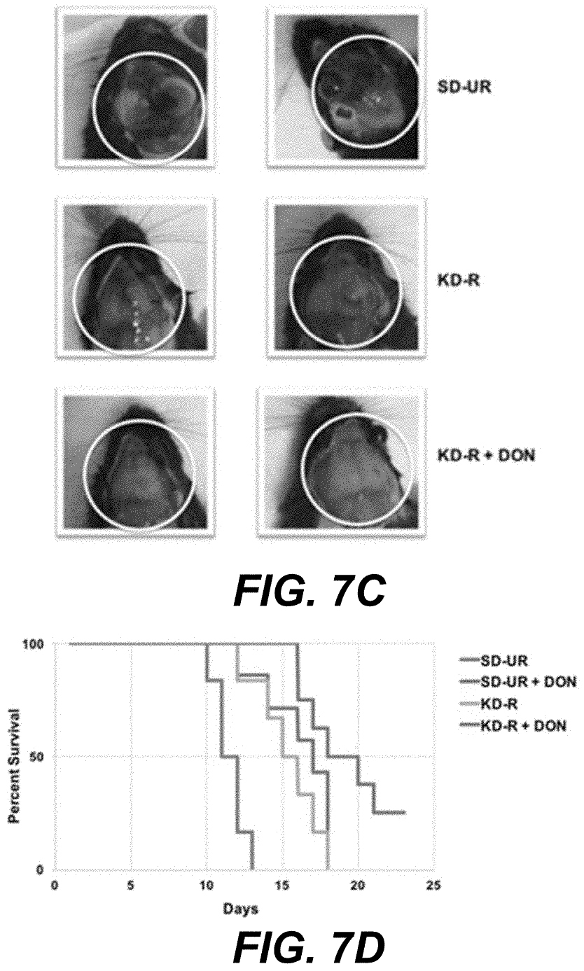

The CT-2A tumour is characterized as a highly angiogenic malignant mouse astrocytoma with stem cell characteristics^{32,38}. The wet weight of the brain plus CT-2A tumour was significantly lower in mice fed the KD-R than in mice fed the SD-UR, indicating a reduction of edema (FIG. 7A). Ex-vivo bioluminescence of brains containing the CT-2A tumour was significantly lower in mice fed the KD-R+DON than in mice fed the SD-UR indicating a significant reduction in the number of living CT-2A tumour cells in the brain (FIG. 7B). Representative mice with the CT-2A brain tumour are shown in FIG. 7C. The brains of mice bearing the CT-2A tumour that were fed the SD-UR were highly hemorrhagic, thrombotic, and edematous. In contrast, the brains of CT-2A bearing mice that were treated with KD-R+DON appeared normal without hemorrhage, thrombosis, or edema (FIG. 7C). All untreated SD-UR control mice containing the CT-2A tumour died between day 10 and 13 (FIG. 7D). In contrast, CT-2A-bearing mice treated with the diet/drug combination survived until day 24 and onwards. Histological analysis also showed that CT-2A tumour cell density, mitotic figures, and hemorrhage was less prevalent in the mice fed the KD-R than in the mice fed the SD-UR (FIG. 11). The appearance of mitotic arrest or catastrophe was also seen in the CT-2A tumour of mice treated with the KD-R+DON (FIG. 11).

### Example 8

### Methods and Experimental Procedures

### Materials and Methods

### Mice

Mice of the VM/Dk (VM) strain were obtained as a gift from H. Fraser (University of Edinburgh, Scotland). The C57BL/6J (B6) mice were obtained originally from the Jackson Laboratory, Bar Harbor, Me. All mice used in this study were housed and bred in the Boston College Animal Care Facility using husbandry conditions as previously described²⁸. All animal procedures and protocols were in strict accordance with the NIH Guide for the Care and Use of Laboratory Animals and were approved by the Institutional Animal Care Committee at Boston College under assurance number A3905-01.

### The VM-M3 and CT-2A Murine GBM Models

The VM-M3 tumour used in this study arose spontaneously in the cerebrum of an adult male mouse of the VM/Dk inbred strain. A cell line was prepared from the tumour as described previously³⁷. The VM-M3 tumour manifests all of the invasive characteristics seen in human GBM². The CT-2A tumour was originally produced from implantation of 20-methylcholanthrene into the cerebral cortex of a C57BL/6J mouse and was broadly classified as a poorly differentiated highly malignant anaplastic astrocytoma and a cell line was produced from this tumour as described³¹. More recent studies have classified the CT-2A tumour as a neural stem cell tumour³². The VM-M3 and the CT-2A cell lines were transduced with a lentivirus vector containing the firefly luciferase gene under control of the cytomegalovirus promoter (VM-M3/Fluc) to produce VM-M3/Fluc and CT-2A/Fluc cell lines, as we previously described (gift from Miguel Sena-Esteves)³⁷. This transduction allows the cells to be tracked in the brain using bioluminescent imaging.

### Tumour Implantation

Tumour implantation was performed as previously described². Briefly mice were anaesthetized with isoflurane (5% in oxygen). The tops of the heads were disinfected with ethanol and a small incision was made in the scalp over the midline. A 3 mm³ burr hole was made in the skull over the right parietal region behind the coronal suture and lateral to the sagittal suture. Small (1 mm³) tumour fragments were implanted approximately 1.5-2.0 mm deep into the cortical region using a trocar, as previously described. The skin flaps were closed with 7 mm reflex clips. The mice were placed in a warm room (24° C.) until they were fully recovered. The procedure confirms 100% recovery within a few hours of implantation. The GBM tumour cells are highly invasive regardless of implantation method, and all tumour-implanted mice reach morbidity at approximately 12-15 days.

### Dietary/Drug Regimens and Body Weight

All mice received the standard diet unrestricted (SD-UR) prior to initiation of the study. Tumour fragments were implanted on day zero. Upon implantation of the tumour, mice remained on the SD-UR until a 15-h fast was initiated 72 h after implantation. Following the fast, mice were introduced to the Ketogenic diet restricted (KD-R), or remained on the SD-UR, depending on the study group. The KD-R has a caloric density of 7.12 Kcal/g. The percent nutritional breakdown of KetoGEN (Medica Nutrition, Canada) is as follows: 2.1% of Cal from carbohydrates, 8.7% of Cal from protein, and 89.2% of Cal from fat (See Table below).

Composition (%) of the standard diet and the ketogenic diet

| | Standard | Ketogenic |
|---|---|---|
| Components | Diet | Diet |
| Carbohydrate | 62 | 3 |
| Fat | 6 | 72 |
| Protein | 27 | 15 |
| Energy | 4.4 | 7.2 |
| (Kcal/g) | | |

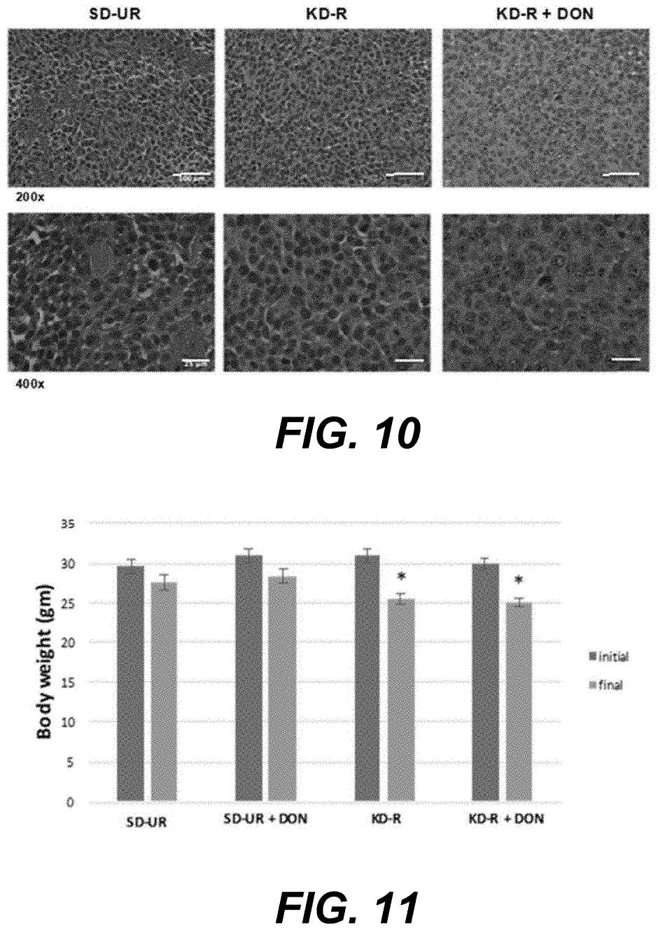

The Medica Nutrition cancer ketogenic diet that we used was prepared without added vitamins and minerals. Previous studies by Tannanbaum showed that the therapeutic effects of calorie restricted diets come largely from the restriction of macronutrients and not from restriction of micronutrients⁷⁰. Mice were individually housed beginning at the initiation of the 15 h fast. Food intake for the KD-R mice was measured to be between 1-3 g per day to maintain a 15-18% body weight reduction in each mouse. Mice were weighed daily to ensure weight maintenance (FIG. 10).

Mice receiving the SD-UR were given the standard chow diet (Lab Diet) ad libitum for the duration of the study. For those mice that received DON injections, a fresh stock was prepared and diluted to an appropriate concentration in PBS and was administered intraperitoneally (i.p.). The DON stock solution in PBS was stored at −20° C. for the duration of the study and mice received 200 μl injections of 0.1-1.0 mg/kg. Mice in the DON survival study received DON at a dosage that was appropriate for the individual mouse response. Some doses were skipped if the mice appeared lethargic or if body weight loss exceeded 1.5 g from the previous day. Studies were terminated at the time of morbidity for the control SD-UR group.

### Bioluminescence Imaging

The Xenogen IVIS system is used to record the bioluminescent signal from the labeled tumours as we previously described³⁷. Briefly, for in-vivo imaging, mice received an i.p. injection of d-lucifierin (50 mg/kg) in PBS and Isofluorane (5% in oxygen). Imaging times ranged from 1 to 5 min, depending on the time point. For ex-vivo imaging, brains were removed and imaged in 0.3 mg d-luciferin in PBS. The IVIS Lumina cooled CCD camera system was used for light acquisition. Data acquisition and analysis was performed with Living Image software (Caliper LS).

### Histology

Brain tumour samples were fixed in 10% neutral buffered formalin (Sigma) and embedded in paraffin. The brain tumour samples were sectioned at 5 μm, were stained with haematoxylin and eosin (H&E) at the Harvard University Rodent Histopathology Core Facility (Boston, Mass.), and were examined by light microscopy using either a Zeiss Axioplan 2 or Nikon SMZ1500 light microscope. Images were acquired using SPOT Imaging Solutions (Diagnostic Instruments, Inc.) cameras and software. All histological sections were evaluated at the Harvard University Rodent Histopathology Core Facility.

### Immunohistochemistry: Ki-67 Proliferation Marker and Iba-1 Microglial Marker

For immunohistochemistry, the tissue sections from untreated and treated tumour bearing mice were deparaffinized, rehydrated, and washed. The tissue sections were then heat treated (95° C.) in antigen unmasking solution (Vector Laboratories, Burlingame, Calif.) for 30 min. Tissue sections were blocked in goat serum (1:10 in PBS) for one hour at room temperature, treated with Ki-67 primary antibody (rat monoclonal, Dako, 1:100) overnight at 4° C. followed by Alexafluor conjugated anti-rat secondary antibody at 1:100 dilution. Nuclei were stained using NucBlue (Invitrogen). Images were captured by the EVOS FL Cell Imaging System fluorescent microscope. For Iba-1, tissue sections were blocked with Iba-1 primary antibody (rabbit monoclonal, abcam, 1:1000) overnight at 4° C. followed by biotinylated anti-rabbit secondary at 1:2000 dilution. Avidin-biotin reaction was complete by incubating the tissues in ABC reagent (Vector Lab) for 30 minutes followed by DAB substrate reaction. Methyl green was used for staining the nucleus.

### Western Blot Analysis of Ib-1 Protein Expression.

Frozen tumour and normal brain tissues were homogenized in ice-cold lysis buffer containing 20 mmol/L Tris-HCl (pH 7.5), 150 mmol/L NaCl, 1 mmol/L Na₂EDTA, 1 mmol/L EGTA, 1% Triton, 2.5 mmol/L NaPPi, 1 mmol/L a-glycerophosphate, 1 mmol/L Na3PO4, 1 Ag/mL leupeptin, and 1 mmol/L phenylmethylsufonyl fluoride. Lysates were transferred to 1.7 mL Eppendorf tubes, mixed on a rocker for 1 h at 4° C., and then centrifuged at 8,100×g for 20 min. Supernatants were collected and protein concentrations were estimated using the Bio-Rad detergent-compatible protein assay.

Approximately 100 μg of total protein from each tissue sample was denatured with SDS-PAGE sample buffer [63 mmol/L Tris-HCl (pH 6.8), 10% glycerol, 2% SDS, 0.0025% bromphenol blue, and 5% 2-mercaptoethanol] and was resolved by SDS-PAGE on 4% to 12% Bis-Tris gels (Invitrogen). Proteins were transferred to a polyvinylidene difluoride immobilon TM-P membrane (Millipore) overnight at 4° C. and blocked in either 5% nonfat powdered milk or 5% bovine serum albumin in TBS with Tween 20 (pH 7.6) for 1 to 3 h at room temperature. Membranes were probed with primary antibodies (Iba-1, abcam, UK) overnight at 4° C. with gentle shaking. The blots were then incubated with the appropriate secondary antibody (anti-rabbit) for 1 h at room temperature and bands were visualized with enhanced chemiluminescence. Each membrane was stripped and reprobed for β-actin as an internal loading control and the ratio of the Iba-1 to β-actin was analyzed by scanning densitometry (FluorChem 8900 Software).

### Liquid Chromatography Mass Spectrometry Analysis of DON

Two independent LC-MS instruments and procedures were used to measure the amount of DON in the blood and in the VM-M3 tumour tissues. This was done to validate the accuracy of the procedures. A Triple quad method (Agilent 6460) was used to analyze DON in the first procedure (FIG. 4A). Mice were injected with 1.0 mg/kg of DON at either 10 min or 60 min before collection of the brains and blood. The brains were immediately flash frozen. Blood samples were collected before removal of the brain. 3N HCl+n-butanol (250 μl) was added to 50 μl of plasma, vortexed and then centrifuged at 16,000×g for 5 min to precipitate proteins. A 200 μl aliquot of the supernatant was transferred to a 1.5 ml tube and incubated at 60° C. for 30 min in a shaking water bath to perform the DON derivatization reaction. For the brain tissue, 5 μl of n-butanol containing 3N HCl was added per milligram tissue. The tissue sample was then homogenized with a glass pestle and was then vortexed and centrifuged, and prepared similarly to the plasma samples. Standard solutions were prepared by serial dilution to generate concentrations from 10 nM to 100 μM. DON was added to untreated mouse plasma or brain tissue to generate an internal standard curve. A high-resolution LC-MS QToF method (Agilent 6550) was used to analyze DON in the second procedure (FIG. 4B). Mice were injected with DON as above. Pre-weighed frozen brain tissue was transferred to a tube containing ceramic beads (Omni tubes). 3M HCl+butanol was added at a concentration of 5 μI/mg of tissue. The sample was then homogenized via a bead ruptor homogenizer for 45 seconds, let rest for 30 seconds, and homogenized again with the bead ruptor for an additional 45 seconds. Subsequently, the homogenate was derivatized by incubating at 60° C. for 30 minutes. The homogenate was then centrifuged at 16,000×g for 10 minutes at 4° C. The supernatant was transferred to a fresh 1.5 ml tube and was centrifuged again to remove excess debris. A 200 μL aliquot of the supernatant was dried in a centrifugal evaporator. The samples were resuspended in 50 μl dH₂O containing 0.2% formic acid. A 5 μl aliquot of this solution was injected for DON analysis using a Phenomenex Kinetix 2.6 μM F5 column for an 8 min gradient. Standard curves were also used as described above.

### Blood Glucose and Ketone Measurements

All mice were fasted for 2 hours before blood collection to stabilize blood glucose levels. Blood glucose and ketone levels were measured using the Keto-Mojo monitoring system (keto-mojo, Napa, Calif.). Whole blood from the tail was placed onto the glucose or ketone strip. The keto-mojo meter was used to determine the mmol levels of glucose and β-hydroxybutyrate in the blood. The Glucose Ketone Index (GKI) was determined as we previously described³⁶.

### Tumour Necrosis Factor (TNF) Determination

VM-M3 brain tumour tissue and normal brain tissue from the VM/Dk mice were homogenized and processed following the Quantikine Mouse TNF-alpha Immunoassay protocol (R&D systems). TNF-alpha levels were measured in triplicate and adjusted with the protein level of each sample. The limit of detection is <10 pg/ml TNF.

### Statistics

Body weight, food intake, tumour growth, and plasma metabolite levels were analyzed using the one-way analysis of variance (ANOVA) followed by Tukey's post hoc test or by a Student's t test to perform a two-sided pairwise comparison among the groups (SPSS 14.0). In each figure, error bars are mean±SEM and n is the number of individual mice analyzed. The Survival studies were plotted on a Kaplan Meir curve using Graph Pad Prizm software and significance was determined using the log-rank test.

### Data Availability

All data supporting the findings presented are available upon request. The source data for all figures are provided as a source data file.

### C. References

References are cited herein throughout using the format of reference number(s) enclosed by parentheses corresponding to one or more of the following numbered references. For example, citation of references numbers 1 and 2 immediately herein below would be indicated in the disclosure as (1, 2) or (1-2).
1 Desjardins, A. et al. Recurrent Glioblastoma Treated with Recombinant Poliovirus. N Engl J Med 379, 150-161, doi:10.1056/NEJMoa1716435 (2018).
2 Shelton, L. M. et al. A novel pre-clinical in vivo mouse model for malignant brain tumor growth and invasion. J Neurooncol 99, 165-176 (2010).
3. Karsy, M. et al. Established and emerging variants of glioblastoma multiforme: review of morphological and molecular features. Folia Neuropathol 50, 301-321 (2012).
4 Huysentruyt, L. C., Akgoc, Z. & Seyfried, T. N. Hypothesis: are neoplastic macrophages/microglia present in glioblastoma multiforme? ASN neuro 3, doi:10.1042/AN20110011 (2011).
5 Yuan, X. et al. Isolation of cancer stem cells from adult glioblastoma multiforme. Oncogene 23, 9392-9400 (2004).
6 Seyfried, T. N., Yu, G., Maroon, J. C. & D'Agostino, D. P. Press-pulse: a novel therapeutic strategy for the metabolic management of cancer. Nutr Metab (Lond) 14, 19, doi:10.1186/s12986-017-0178-2 (2017).
7 Feichtinger, R. G. et al. Alterations of oxidative phosphorylation complexes in astrocytomas. Glia 62, 514-525, doi:10.1002/glia.22621 (2014).
8 Arismendi-Morillo, G. J. & Castellano-Ramirez, A. V. Ultrastructural mitochondrial pathology in human astrocytic tumors: potentials implications pro-therapeutics strategies. J Electron Microsc (Tokyo) 57, 33-39 (2008).
9 Katsetos, C. D., Anni, H. & Draber, P. Mitochondrial dysfunction in gliomas. Seminars in pediatric neurology 20, 216-227, doi:10.1016/j.spen.2013.09.003 (2013).
10 Deighton, R. F. et al. Interactions among mitochondrial proteins altered in glioblastoma. Journal of neuro-oncology 118, 247-256, doi: 10.1007/s11060-014-1430-5 (2014).
11 Arismendi-Morillo, G., Castellano-Ramirez, A. & Seyfried, T. N.
Ultrastructural characterization of the Mitochondria-associated membranes abnormalities in human astrocytomas: Functional and therapeutics implications. Ultrastruct Pathol 41, 234-244, doi:10.1080/01913123.2017.1300618 (2017).
12 Kiebish, M. A., Han, X., Cheng, H., Chuang, J. H. & Seyfried, T. N. Cardiolipin and electron transport chain abnormalities in mouse brain tumor mitochondria: lipidomic evidence supporting the Warburg theory of cancer. Journal of lipid research 49, 2545-2556 (2008).
13 Lehninger, A. L. The Mitochondrion: Molecular Basis of Structure and Function. (W. A. Benjamin, INC., 1964).
14 Bartesaghi, S. et al. Inhibition of oxidative metabolism leads to p53 genetic inactivation and transformation in neural stem cells. Proceedings of the National Academy of Sciences of the United States of America 112, 1059-1064, doi:10.1073/pnas.1413165112 (2015).
15 Elsakka, A. M. A. et al. Management of Glioblastoma Multiforme in a Patient Treated With Ketogenic Metabolic Therapy and Modified Standard of Care: A 24-Month Follow-Up. Front Nutr 5, 20, doi:10.3389/fnut.2018.00020 (2018).
16 Oizel, K. et al. Efficient Mitochondrial Glutamine Targeting Prevails Over Glioblastoma Metabolic Plasticity. Clin Cancer Res 23, 6292-6304, doi:10.1158/1078-0432.CCR-16-3102 (2017).
17 Liu, Z. et al. MicroRNA-153 regulates glutamine metabolism in glioblastoma through targeting glutaminase. Tumour Biol 39, 1010428317691429, doi:10.1177/1010428317691429 (2017).
18 Rhodes, C. G. et al. In vivo disturbance of the oxidative metabolism of glucose in human cerebral gliomas. Ann Neurol 14, 614-626 (1983).
19 Yang, C. et al. Glioblastoma Cells Require Glutamate Dehydrogenase to Survive Impairments of Glucose Metabolism or Akt Signaling. Cancer research (2009).
20 Seyfried, T. N., Flores, R., Poff, A. M., D'Agostino, D. P. & Mukherjee, P. Metabolic therapy: a new paradigm for managing malignant brain cancer. Cancer letters 356, 289-300, doi:10.1016/j.canlet.2014.07.015 (2015).
21 Seyfried, T. N. & Mukherjee, P. Targeting energy metabolism in brain cancer: review and hypothesis. Nutr Metab (Lond) 2, 30 (2005).
22 Seyfried, T. N., Shelton, L. M. & Mukherjee, P. Does the existing standard of care increase glioblastoma energy metabolism? Lancet Oncol 11, 811-813, doi:10.1016/S1470-2045(10)70166-2 (2010).
23 Zhou, W. et al. The calorically restricted ketogenic diet, an effective alternative therapy for malignant brain cancer. Nutr Metab (Lond) 4, 5 (2007).
24 Kim, D. Y. et al. Ketone bodies are protective against oxidative stress in neocortical neurons. Journal of neurochemistry (2007).
25 Chinopoulos, C. & Seyfried, T. N. Mitochondrial substrate level phosphorylation as energy source for glioblastoma: Review and hypothesis. ASN neuro 10, 1-27 (2018).
26 Flores, R. E. et al. Mycoplasma infection and hypoxia initiate succinate accumulation and release in the VM-M3 cancer cells. Biochimica et biophysica acta, doi:10.1016/j.bbabio.2018.03.012 (2018).
27 Souba, W. W. Glutamine and cancer. Ann Surg 218, 715-728 (1993).
28 Shelton, L. M., Huysentruyt, L. C. & Seyfried, T. N. Glutamine targeting inhibits systemic metastasis in the VM-M3 murine tumor model. Inter. J. Cancer. 127, 2478-2485, doi:10.1002/ijc.25431 (2010).
29 Cervantes-Madrid, D., Romero, Y. & Duenas-Gonzalez, A.
Reviving Lonidamine and 6-Diazo-5-oxo-L-norleucine to Be Used in Combination for Metabolic Cancer Therapy. Biomed Res Int 2015, 690492, doi:10.1155/2015/690492 (2015).
30 Lemberg, K. M., Vornov, J. J., Rais, R. & Slusher, B. S. We're Not “DON” Yet: Optimal Dosing and Prodrug Delivery of 6-Diazo-5-oxo-L-norleucine. Mol Cancer Ther 17, 1824-1832, doi:10.1158/1535-7163.MCT-17-1148 (2018).
31 Seyfried, T. N., el-Abbadi, M. & Roy, M. L. Ganglioside distribution in murine neural tumors. Mol Chem Neuropathol 17, 147-167 (1992).
32 Binello, E., Qadeer, Z. A., Kothari, H. P., Emdad, L. & Germano, I. M. Stemness of the CT-2A Immunocompetent Mouse Brain Tumor Model: Characterization In Vitro. Journal of Cancer 3, 166-174, doi:10.7150/jca.4149 (2012).
33 Gordon, E. B. et al. Targeting glutamine metabolism rescues mice from late-stage cerebral malaria. Proceedings of the National Academy of Sciences of the United States of America 112, 13075-13080, doi:10.1073/pnas.1516544112 (2015).
34 Seyfried, T. N., Sanderson, T. M., El-Abbadi, M. M., McGowan, R. & Mukherjee, P. Role of glucose and ketone bodies in the metabolic control of experimental brain cancer. Br J Cancer 89, 1375-1382 (2003).
35 Meidenbauer, J. J., Ta, N. & Seyfried, T. N. Influence of a ketogenic diet, fish-oil, and calorie restriction on plasma metabolites and lipids in C57BL/6J mice. Nutrition & metabolism 11, 23, doi:10.1186/1743-7075-11-23 (2014).
36 Meidenbauer, J. J., Mukherjee, P. & Seyfried, T. N. The glucose ketone index calculator: a simple tool to monitor therapeutic efficacy for metabolic management of brain cancer. Nutr Metab (Lond) 12, 12, doi:10.1186/s12986-015-0009-2 (2015).
37 Huysentruyt, L. C., Mukherjee, P., Banerjee, D., Shelton, L. M. & Seyfried, T. N. Metastatic cancer cells with macrophage properties: evidence from a new murine tumor model. Int J Cancer 123, 73-84 (2008).
38 Mukherjee, P., Abate, L. E. & Seyfried, T. N. Antiangiogenic and proapoptotic effects of dietary restriction on experimental mouse and human brain tumors. Clin Cancer Res 10, 5622-5629 (2004).
39 Duran, R. V. & Hall, M. N. Glutaminolysis feeds mTORC1. Cell Cycle 11, 4107-4108, doi:10.4161/cc.22632 (2012).
40 Roberts, M. N. et al. A Ketogenic Diet Extends Longevity and Healthspan in Adult Mice. Cell Metab 26, 539-546 e535, doi:10.1016/j.cmet.2017.08.005 (2017).
41 Nebeling, L. C., Miraldi, F., Shurin, S. B. & Lerner, E. Effects of a ketogenic diet on tumor metabolism and nutritional status in pediatric oncology patients: two case reports. J. Am. Coll. Nutr. 14, 202-208 (1995).
42 Newsholme, P. Why is L-glutamine metabolism important to cells of the immune system in health, postinjury, surgery or infection? J Nutr 131, 2515S-2522S; discussion 2523S-2514S (2001).
43 Denny, C. A. et al. Restricted ketogenic diet enhances the therapeutic action of N-butyldeoxynojirimycin towards brain GM2 accumulation in adult Sandhoff disease mice. Journal of neurochemistry 113, 1525-1535 (2010).
44 Urits, I., Mukherjee, P., Meidenbauer, J. & Seyfried, T. N. Dietary restriction promotes vessel maturation in a mouse astrocytoma. J. Oncology Article ID 264039, 10 (2012).
45 Amores-Sanchez, M. I. & Medina, M. A. Glutamine, as a precursor of glutathione, and oxidative stress. Mol Genet Metab 67, 100-105, doi:10.1006/mgme.1999.2857 (1999).
46 Medina, M. A. & Nunez de Castro, I. Glutaminolysis and glycolysis interactions in proliferant cells. Int J Biochem 22, 681-683 (1990).
47 Galarraga, J. et al. Glucose metabolism in human gliomas: correspondence of in situ and in vitro metabolic rates and altered energy metabolism. Metab Brain Dis 1, 279-291. (1986).
48 Coleman, M. T. & Allen, N. The hexose monophosphate pathway in ethylnitrosourea induced tumors of the nervous system. Journal of neurochemistry 30, 83-90 (1978).
49 Libby, C. J., Tran, A. N., Scott, S. E., Griguer, C. & Hjelmeland, A. B. The pro-tumorigenic effects of metabolic alterations in glioblastoma including brain tumor initiating cells. Biochimica et biophysica acta 1869, 175-188, doi:10.1016/j.bbcan.2018.01.004 (2018).
50 Marsh, J., Mukherjee, P. & Seyfried, T. N. Akt-dependent proapoptotic effects of dietary restriction on late-stage management of a phosphatase and tensin homologue/tuberous sclerosis complex 2-deficient mouse astrocytoma. Clin Cancer Res 14, 7751-7762 (2008).
51 Mulrooney, T. J., Marsh, J., Urits, I., Seyfried, T. N. & Mukherjee,
P. Influence of Caloric Restriction on Constitutive Expression of NF-kappaB in an Experimental Mouse Astrocytoma. PloS one 6, e18085 (2011).
52 Dogan, S., Johannsen, A. C., Grande, J. P. & Cleary, M. P. Effects of intermittent and chronic calorie restriction on mammalian target of rapamycin (mTOR) and IGF-I signaling pathways in mammary fat pad tissues and mammary tumors. Nutr Cancer 63, 389-401, doi:10.1080/01635581.2011.535968 (2011).
53 Yang, M. & Vousden, K. H. Serine and one-carbon metabolism in cancer. Nat Rev Cancer 16, 650-662, doi:10.1038/nrc.2016.81 (2016).
54 Veech, R. L. The therapeutic implications of ketone bodies: the effects of ketone bodies in pathological conditions: ketosis, ketogenic diet, redox states, insulin resistance, and mitochondrial metabolism. Prostaglandins Leukot Essent Fatty Acids 70, 309-319 (2004).
55 Cahill, G. F., Jr. &Veech, R. L. Ketoacids? Good medicine? Trans Am Clin Climatol Assoc 114, 149-161; discussion 162-143 (2003).
56 Maurer, G. D. et al. Differential utilization of ketone bodies by neurons and glioma cell lines: a rationale for ketogenic diet as experimental glioma therapy. BMC cancer 11, 315, doi:10.1186/1471-2407-11-315 (2011).
57 Bartmann, C. et al. Beta-hydroxybutyrate (3-OHB) can influence the energetic phenotype of breast cancer cells, but does not impact their proliferation and the response to chemotherapy or radiation. Cancer & metabolism 6, 8, doi:10.1186/s40170-018-0180-9 (2018).
58 Duran, R. V. et al. Glutaminolysis activates Rag-mTORC1 signaling. Mol Cell 47, 349-358, doi:10.1016/j.molcel.2012.05.043 (2012).
59 Medina, M. A. Glutamine and cancer. J Nutr 131, 2539S-2542S; discussion 2550S-2531S (2001).
60 Fendt, S. M. et al. Reductive glutamine metabolism is a function of the alpha-ketoglutarate to citrate ratio in cells. Nature communications 4, 2236, doi:10.1038/ncomms3236 (2013).
61 Kim, M. H. & Kim, H. The Roles of Glutamine in the Intestine and Its Implication in Intestinal Diseases. Int J Mol Sci 18, doi:10.3390/ijms18051051 (2017).
62 Winter, S. F., Loebel, F. & Dietrich, J. Role of ketogenic metabolic therapy in malignant glioma: A systematic review. Crit Rev Oncol Hematol 112, 41-58, doi:10.1016/j.critrevonc.2017.02.016 (2017).
63 Schwartz, K. A., Noel, M., Nikolai, M. & Chang, H. T. Investigating the Ketogenic Diet As Treatment for Primary Aggressive Brain Cancer: Challenges and Lessons Learned. Front Nutr 5, 11, doi:10.3389/fnut.2018.00011 (2018).
64 Shelton, L. M., Huysentruyt, L. C., Mukherjee, P. & Seyfried, T. N. Calorie restriction as an anti-invasive therapy for malignant brain cancer in the VM mouse. ASN neuro 2, e00038 (2010).
65 Schwartzbaum, J. et al. Associations between prediagnostic blood glucose levels, diabetes, and glioma. Sci Rep 7, 1436, doi:10.1038/s41598-017-01553-2 (2017).
66 Biancur, D. E. et al. Compensatory metabolic networks in pancreatic cancers upon perturbation of glutamine metabolism. Nat Commun 8, 15965, doi:10.1038/ncomm515965 (2017).
67 Marin-Valencia, I. et al. Analysis of tumor metabolism reveals mitochondrial glucose oxidation in genetically diverse human glioblastomas in the mouse brain in vivo. Cell metabolism 15, 827-837, doi:10.1016/j.cmet.2012.05.001 (2012).
68 Mashimo, T. et al. Acetate is a bioenergetic substrate for human glioblastoma and brain metastases. Cell 159, 1603-1614, doi:10.1016/j.cell.2014.11.025 (2014).
69 Tardito, S. et al. Glutamine synthetase activity fuels nucleotide biosynthesis and supports growth of glutamine-restricted glioblastoma. Nat Cell Biol 17, 1556-1568, doi:10.1038/ncb3272 (2015).
70 Tannenbaum, A. The genesis and growth of tumors: II. Effects of caloric restriction per se. Cancer research 2, 460-467 (1942).

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A diet-drug combination for treating cancer comprising a calorie restricted diet and a drug that targets one or more energy metabolite of cancel cells for growth and progression.

2. The combination of claim 1, wherein the calorie restricted diet is ketogenic diet (KD-R).

3. The combination of claim 1, wherein the drug is a glucose or glutamine analogue.

4. The combination of claim 3, where the glutamine analogue is 6-diazo-5-oxo-L-norleucine (DON).

5. The combination of claim 1, wherein the energy metabolite comprises glucose, glutamine, or combinations thereof.

6. The combination of claim 1, wherein the cancer is the cancer uses glucose, glutamine, or combination of energy metabolties thereof, for cancer cell growth and progression.

7. The combination of claim 6, wherein the cancer is brain cancer, breast cancer, lung cancer, skin cancer, kidney cancer or pancreatic cancer.

8. The combination of claim 7, wherein the brain cancer is glioblastoma (GBM).

9. The combination of claim 8, wherein the glioblastoma (GBM) is human glioblastoma multiforme.

10. The combination of claim 6, wherein the cancer is invasive mesenchymal tumour or stem cell glioma.

11. A method of managing cancer, comprising the step of a) placing a patient in need on a calorie restricted diet, and b) administering an effective amount of a drug that targets one or more energy metabolite of cancel cells for growth and progression.

12. The method of claim 11, wherein the calorie restricted diet is ketogenic diet (KD-R).

13. The method of claim 11, wherein the drug is a glucose or glutamine analogue.

14. The method of claim 13, wherein the glutamine analogue is 6-diazo-5-oxo-L-norleucine (DON).

15. The method of claim 11, wherein the energy metabolite comprises glucose, glutamine, or combinations thereof.

16. The method of claim 11, wherein the cancer is the cancer uses glucose, glutamine, or combination of energy metabolties thereof, for cancer cell growth and progression.

17. The method of claim 11, wherein the cancer is brain cancer, breast cancer, lung cancer, skin cancer, kidney cancer or pancreatic cancer.

18. The method of claim 17, wherein the brain cancer is glioblastoma (GBM).

19. The method of claim 18, wherein the glioblastoma (GBM) is human glioblastoma multiforme.

20. The method of claim 16, wherein the cancer is invasive mesenchymal tumour or stem cell glioma.
